(19) United States
(12) Patent Application Publication
(10) Pub. No.: US 2018/0256049 A1
(43) Pub. Date: Sep. 13, 2018

(54) Heart Rate Monitor

(71) Applicant: TomTom International B.V., Amsterdam (NL)

(72) Inventors: Stephen Bayley, Amsterdam (NL); Clement Albert Anne Magniez, Amsterdam (NL); Stephen Michael Jackson, Amsterdam (NL)

(21) Appl. No.: 15/756,970

(22) PCT Filed: Sep. 2, 2016
(86) PCT No.: PCT/EP2016/070734

§ 371 Date: Mar. 1, 2018

(30) Foreign Application Priority Data
Sep. 3, 2015 (GB) 1515657.3

(51) Int. Cl. A61B 5/024; A61B 5/00

(52) U.S. Cl. CPC A61B 5/02438; A61B 5/742; A61B 5/6831; A61B 5/02427; A61B 5/6824; A61B 5/681

(57) ABSTRACT

A heart rate monitor comprises a housing (30) and an optical heart rate (OHR) sensor within the housing (30). The OHR sensor comprises a sensing unit (40) including at least one light emitter (42a, 42b) arranged to emit light into the skin of a user and a photodetector (44) arranged to sense light reflected through the skin of the user. The housing (30) comprises a domed portion (60). The sensing unit (40) is exposed through a surface of the domed portion (60) and protrudes above the surface of the domed portion (60).

### FIELD OF THE INVENTION

This invention relates to heart rate monitors, and more specifically to heart rate monitors comprising an optical heart rate (OHR) sensor. Furthermore the invention relates to a wearable heart rate monitor comprising an OHR sensor and to the mounting of such a monitor using a wrist strap. The heart rate monitor may be provided as a fitness watch, for example with the heart rate monitor is removably mounted to a wrist strap. Illustrative embodiments of the invention relate to devices for monitoring athletic performance, e.g. that can be worn during an exercise activity (running, cycling, swimming, hiking, skiing, weightlifting, etc.), which can track, display and record the heart rate of the user at particular moments during a workout.

### BACKGROUND OF THE INVENTION

Conventional heart rate monitors typically take the form of a sensor mounted to a chest strap. The capacitive sensor detects the heart's electrical activity through the skin. This requires the chest strap to mount the sensor close to the heart and to hold it firmly against the skin. A user wears the chest strap and a corresponding device, such as a wrist watch, which is wirelessly linked to the heart rate sensor to receive and display heart rate information. Alternatively the sensor mounted to the chest strap might be linked to a non-wearable display device such as a mobile phone or a treadmill controller.

Alternatively, some heart rate monitors can be worn on the wrist rather than using a chest strap. These pulse monitors require a user to touch a finger against a pad to provide a pulse rate. However this type of monitor requires the user to stop exercising to take the pulse reading and tends to be less accurate than the chest strap monitors.

More recently, strapless heart rate monitors have taken the form of wrist-worn devices that use optical sensing of the volume of blood under the skin. WO 2013/042070 A1 discloses an optical heart rate monitor that includes a housing in the form of a watch for wearing on the wrist or arm of a user. The monitor comprises an LED arranged to emit light into the skin of a user, where it is partially absorbed by the underlying blood vessels, and a photodetector arranged to sense light reflected back through the skin. The monitor processes the sensor signals and determines a pulse and/or heart rate for display.

Optical heart rate monitors must be mounted in stable contact with the skin during the use to avoid motion artefacts and ensure reliable measurements. Furthermore, ambient light artefacts can reduce measurement quality, for example when a user moves between locations of different ambient lighting, e.g. between shadow and direct sunlight. In WO 2013/042070 A1 an optical high-pass filter is used when detecting the reflected light signals so as to filter out ambient infrared light.

It is desired, in at least embodiments of the present invention, to provide an improved heart rate monitor.

### SUMMARY OF THE INVENTION

A first aspect of the present invention provides a heart rate monitor comprising a housing and an optical heart rate (OHR) sensor within the housing, the OHR sensor comprising a sensing unit including at least one light emitter arranged to emit light into the skin of a user and a photodetector arranged to sense light reflected through the skin of the user, wherein the housing comprises a domed portion, the sensing unit being exposed through a surface of the domed portion, and wherein the sensing unit protrudes above the surface of the domed portion.

According to the present invention, the housing comprises a domed portion that acts to press against the skin of a user when the monitor is worn during use. This helps to form a good area of contact against the skin. Furthermore, because the sensing unit protrudes above the surface of the domed portion, the sensing unit may press slightly into the wearer's skin so that substantially no ambient light can reach the photodetector. As a result, the photodetector may not need to include an optical filter to remove light artefacts. Furthermore, it is believed that such a structure may also have the dual effect of preventing or reducing motion artefacts by helping to hold the sensing unit in a fixed position against the skin even when a user is moving during exercise. This is particularly suitable when a strap is used to hold the monitor against the surface of a curved limb such as an arm or leg, for example a wrist strap. The present invention is therefore able to overcome the problems outlined above.

The applicant has found that the protrusion of the sensing unit preferably has a minimum of 0.1 mm so as to ensure good contact with the skin of a wearer. Thus in preferred embodiments the sensing unit protrudes above the surface of the domed portion by at least 0.1 mm. The applicant has appreciated that it is desirable for the protrusion of the sensing unit to be limited so that the heart rate monitor is not uncomfortable to wear. Preferably the sensing unit protrudes above the surface of the domed portion by up to 0.8 mm and further preferably up to 0.5 mm.

The depth of the sensing unit may be increased so as to achieve its protrusion. However this may require a bespoke package to be manufactured for the sensing unit. In a preferred set of embodiments the sensing unit is mounted on a circuit board by a riser. By providing a riser between the circuit board and the sensing unit, a standard sensing unit package may be used.

The applicant has recognised that a potential issue with the protrusion of the sensing unit beyond the surface of the housing is an increased risk of damage caused by the ingress of moisture or dirt. This may particularly be a problem in embodiments where the heart rate monitor is provided as a fitness watch and the sensing unit is therefore likely to be assaulted by sweat and/or contaminants (water, dirt, etc.) from the external environment. In a preferred set of embodiments the heart rate monitor further comprises a sealant around the sensing unit. In such examples the sensing unit may be exposed through an aperture in the surface of the domed portion and the sealant may fill the aperture. Suitable sealants may include silicone or polyurethane. In preferred examples the sealant may comprise epoxy resin.

The OHR sensor may comprise any suitable sensing unit including one, two, three or more light emitters. The light emitters preferably comprise light emitting diodes (LEDs). In a preferred set of embodiments, the sensing unit comprises at least two, and preferably three, light emitting diodes of different wavelengths. A suitable OHR sensor is, for example, Osram BioMon SFH7050. This sensor features three LEDs—green (535 nm), red (660 nm) and IR (940 nm)—and a large area photodiode to maximize signal level. The infrared LED can advantageously be used as a proximity sensor to indicate when the sensing unit is in contact with skin. This allows the OHR sensor to automatically start measurements when the monitor is mounted to the skin or to display an out of reach message. Although it is sufficient to drive only one LED (e.g. green) for heart rate monitoring, for pulse oximetry applications the red and infrared LEDs may be driven alternately.

While enjoying the benefits of a sensing unit that protrudes slightly from the housing of the heart rate monitor, it is preferable that the OHR sensor is compact so that the heart rate monitor can also be made thin. This is particularly advantageous for a wrist-worn monitor. In a preferred set of embodiments the sensing unit is fully integrated as a package having a depth of less than 1 mm.

There will now be described some general features of the heart rate monitor that may be combined with one or more of the embodiments outlined above.

The housing of the heart rate monitor is preferably configured as a single integral casing, and which is preferably sealed so as to be water resistant, to allow the monitor to be used for wet weather outdoor exercise and for swimming.

The OHR sensor may comprise a processor and a battery. The processor may be arranged to analyse light signals received, e.g. at the photodetector, for the purposes of display and/or transmission. The OHR sensor may comprise a memory connected to the processor. This means that HR data can be stored by the device and downloaded later. The OHR sensor may comprise an input/output (I/O) device for transferring data to and from the device and for providing power to recharge the battery. In some examples the OHR sensor may simply act as a sensor hub, collecting and/or transmitting heart rate data for display by another device. This may allow the heart rate monitor to be minimised in size.

In a preferred set of embodiments, the heart rate monitor comprises a display for displaying heart rate information to a user. The display may, for example, comprise a liquid crystal display (LCD). Further preferably the heart rate monitor comprises an input device for controlling the OHR sensor and/or the display. Such an input device can enable a user to change relevant functions, such as applicable HR zones. In various embodiments the input device is spaced apart from the display. In embodiments where the monitor is mounted to a wrist strap, the input device is preferably spaced apart from the display in a longitudinal direction of the strap. The display may be configured to display alphanumeric characters or icons such that upper parts of the characters or icons are arranged towards a first side of the housing and the lower parts of the characters or icons are arranged towards a second, opposite side of the housing. The input device is preferably spaced apart from the display in a direction from said first side to said second side. This configuration is useful when a user wears the display on the back of the wrist, as the user is easily able to view the display whilst controlling the device via the input device that is spaced apart from the display. Less preferably, the input device may be spaced apart from the display in a direction from said second side to said first side of the housing. This configuration may be useful, for example, when the monitor is strapped to the handle bars of a bicycle or strapped to another vehicle, as the display can be directed towards the user whilst the user has easy access to the input device from above.

The input device is preferably configured to control the display and associated electrical components in use. For example, the input device may be configured for navigating through a menu displayed on the display. For example, the input device may control the functioning of the OHR sensor. The input device is therefore electrically connected to electronic components in the housing. For example, a ribbon lead may extend between the housing and the input device.

The input device preferably has a substantially planar surface arranged substantially parallel to and above an upper surface of the module. The input device is preferably configured to detect the movement of a user's finger across the substantially planar surface so as to provide an input to control the monitor, e.g. for navigating a menu displayed on the display.

The input device may therefore comprise a touchpad (or trackpad) utilising, for example, capacitive sensing to conductance sensing to translate the motion of a user's finger into an input to control the OHR sensor. The touchpad may comprise a one-dimensional touchpad, and which is capable of sensing motion along a single axis, e.g. left-right or up-down. In other more preferred embodiments, the touchpad may comprise a two-dimensional touchpad, and which is capable of sensing motion in any direction, or at least left-right and up-down, on the plane defined by the substantially planar surface of the input device. In other, albeit less preferred embodiments, the input device may comprise a pointing stick (or trackpad) that senses the force applied by a user's finger, e.g. by using a pair of resistive strain gauges, and translates it into an input to control the monitor.

Alternatively, the input device may comprise a two-way button having a continuous pressing surface and two actuators, the button being configured such that when a first portion of the pressing surface is depressed a first of said actuators is actuated so as to provide a first input to control the module, and when a second portion of the pressing surface is depressed a second of said actuators is actuated so as to provide a second input to control the monitor.

Alternatively, the input device may comprise a four-way button having a continuous pressing surface and four actuators, the button being configured such that when a first portion of the pressing surface is depressed a first of said actuators is actuated so as to provide a first input to control the monitor, when a second portion of the pressing surface is depressed a second of said actuators is actuated so as to provide a second input to control the monitor, when a third portion of the pressing surface is depressed a third of said actuators is actuated so as to provide a third input to control the monitor, and when a fourth portion of the pressing surface is depressed a fourth of said actuators is actuated so as to provide a fourth input to control the monitor. The pressing surface described herein is preferably a substantially planar surface parallel to and above a portion of a lower surface that contacts a user's limb in use. It is also contemplated that the input device may comprise any one or more mechanically actuated buttons or non-mechanically actuated buttons, such as virtual buttons on a touch-sensitive user interface, as desired.

The input device is preferably additionally, or alternatively, configured to be operated by being pressed in a direction that is substantially perpendicular to its substantially planar surface, in a direction from the upper surface towards the lower surface. This enables the user to use a single finger to operate the input device. The user does not need to use a second finger of the same hand to counter-balance the pressing of the input device, because the input device is arranged such that it is pressed against the wrist of the user wearing the monitor.

In preferred embodiments in which the input device is configured to both detect the movement of a user's finger across the substantially planar surface and be pressed against the limb of the user, e.g. where the input device comprises a depressible touch pad, the detected motion of the user's finger is used to navigate a menu for identifying a function to be selected, and the depression of the input device is used to select the identified function.

In addition, or alternatively, the display is preferably substantially planar and arranged in a first plane and the input device has a substantially planar pressing surface arranged in a second plane, wherein the first and second planes are at angles to each other, wherein the angle between the first and second planes is less than 90 degrees, optionally between 20 and 70 degrees. In other words, the planes are imaginary intersecting planes and the sides of the planes facing the user's arm or wrist in use define an angle between them at the intersection, wherein the angle is preferably greater than 90 degrees and less than 180 degree. By providing the surfaces at an angle to each other, the user is enabled a good viewing angle of the display whilst operating the input device, when the monitor is mounted to a user's wrist in use. As the input device is spaced away from the display housing, and hence away from the back of the user's wrist and around the side of the wrist in use, said angle also enables the input device to be orientated such that when it is pressed it is pressed against the user's wrist such that the user's wrist provides the counter-force necessary to balance the pressing force. The input device is therefore able to be operated with a single finger and without needing a second finger on the same hand to counter-balance the pressing force as in conventional watches having buttons around the periphery of the display.

In addition, or alternatively, the display preferably has a casing that is physically connected to the input device by a connecting portion, wherein the connecting portion is curved or angled along a direction from the display to the input device. The connecting portion may be curved or angled such that, when the display is arranged on top of a user's wrist in use, the connecting portion curves or otherwise extends around the wrist such that the input device is located on a side of the user's wrist. The monitor is preferably configured such that the input device is located on the medial side of the user's wrist when the display is located on the back of the wrist, the medial side being the side facing the user's body when the back of the hand is facing vertically upwards. In other less preferred embodiments a wrist strap may form said connecting portion that connects the display casing and the input device. The strap may be flexible or formed from one or more pivotable sections so as to flex or pivot to form the curved or angled connecting portion.

The heart rate monitor preferably comprises a processor configured to control the OHR sensor and the display. The display may visually display heart rate (HR) information such as one or more of: current HR (bpm), average HR (bpm), maximum HR, minimum HR; current HR zone; a graphical representation of HR changes over time; and a graphical representation of the proportion of time spent in each of a plurality of HR zones over time. In addition, or alternatively, the heart rate monitor may comprise an audio output, e.g. a beeper, and/or a haptic output, e.g. a vibrator, to alert a user to changes in the HR data.

The present invention also provides a fitness watch comprising a heart rate monitor as described above. In other words, the heart rate monitor may take the form of a fitness watch and every reference herein to a heart rate monitor may alternatively be taken as a reference to a fitness watch. Such a fitness watch preferably comprises a processor for controlling the heart rate monitor and any other components of the watch. The processor may be connected to means for tracking the location of a user as he or she moves from one location to another, e.g. by using information received from global navigation satellite signals, or by accessing and receiving information from WiFi access points or cellular communication networks. In preferred embodiments the watch comprises a global navigation satellite system (GNSS) receiver, such as a GPS and/or GLONASS receiver, for receiving satellite signals indicating the position, and optionally speed, of the receiver (and thus user) at a particular point in time, and which receives updated information at regular intervals. As will be appreciated, this adds the functionality of tracking the location of the user as he or she moves from one location to another. The GNSS receiver may comprise an antenna, e.g. in the form of a patch antenna, for use in determining the location and movements of the user.

Alternatively, or in addition, the fitness watch may further comprise one or more of: a GPS receiver, a speed sensor, a cadence sensor, an accelerometer, a gyroscope, an altimeter, a pressure sensor (e.g. diving depth gauge), an electronic compass, vibration device for indicating alerts to a user, a wireless communications device (for example capable of transmitting signals from one or more body-worn sensors), such as a Bluetooth module (e.g. capable of using the Bluetooth Low Energy (BLE) protocol). In embodiments where the watch comprises a wireless communications device, this may be arranged to receive data from other sensors, such as a foot pod sensor or a speed/cadence sensor. The wireless communications device may be arranged to communicate with an external heart rate monitor, for example a monitor mounted on a chest strap worn by the user. In addition, or alternatively, the wireless communications device may be arranged to transmit data to one or more external devices (e.g. a mobile phone device).

The watch may comprise one or more electrical connectors for electrically connecting to a dock or cable for charging the battery and/or for transferring data to or from the processor. It is contemplated that any known electrical connector may be employed. In preferred embodiments, however, the one or more electrical connectors comprise electrical contacts, which may be flat and arranged substantially in line with, or recessed in, the lower surface of the housing (e.g. for contacting with corresponding pogo pins in a docking system). The electrical contacts may be located in any portion of the lower surface of the housing as desired, although in preferred embodiments the electrical contacts are located in the lower surface under the input device, e.g. distal from the display. This allows the user to see the display when the watch is positioned in a docking system.

In at least some embodiments, the heart rate monitor may be permanently mounted to a strap to form a fitness watch. For example, the heart rate monitor may be integrated with the strap. However, in various embodiments of the present invention, the heart rate monitor may be removably mounted to a wrist strap. For example, the strap may comprise a central mount to which the heart rate monitor is removably connected. This can allow the heart rate monitor to be repeatedly engaged and disengaged from the strap, for example so that a user can dock the heart rate monitor to allow for the transfer of power and/or data, e.g. using a docking station connected to a computer. In addition, or alternatively, the same strap may be used interchangeably to mount different heart rate monitors or other units, such as a watch module including location determining means, e.g. a global navigation satellite system (GNSS) receiver, such as GPS and/or GLONASS.

The central mount provided by the strap may comprise a physical connector for the heart rate monitor, for example a mechanical and/or magnetic connection system. In a preferred set of embodiments the central mount comprises an aperture in the strap, e.g. into which the heart rate monitor can be inserted. The heart rate monitor may comprise one or more projections and/or recesses for releasably engaging with corresponding features of the aperture. Preferably the strap comprises at least two apertures and, in embodiments where the heart rate monitor comprises a display and an input device, the display and input device each project through a respective aperture in the strap.

The present invention in accordance with any of its further aspects or embodiments may include any of the features described in reference to other aspects or embodiments of the invention to the extent it is not mutually inconsistent therewith.

Advantages of these embodiments are set out hereafter, and further details and features of each of these embodiments are defined in the accompanying dependent claims and elsewhere in the following detailed description.

### BRIEF DESCRIPTION OF THE DRAWINGS

Various aspects of the teachings of the present invention, and arrangements embodying those teachings, will hereafter be described by way of illustrative example with reference to the accompanying drawings, in which:

FIG. 1 shows a perspective view of a heart rate monitor module;

FIG. 2 shows the module of FIG. 1 as viewed from the underside;

FIGS. 3a and 3b show, respectively, a side sectional view and a close-up of the module; and

FIGS. 4a and 4b show a side view and a close-up, respectively, of the module;

FIG. 5 is a schematic illustration of electronic components of a fitness watch according to a preferred embodiment; and

FIG. 6 is a schematic illustration of the manner in which a fitness watch may receive information over a wireless communication channel.

### DETAILED DESCRIPTION OF THE PREFERRED EMBODIMENTS

Preferred embodiments of the present invention will now be described with particular reference to a fitness or sports watch having access to Global Positioning System (GPS) data. Fitness or sports watches of the type described are often worn by athletes to help them during their runs or workouts, e.g. by monitoring the speed and distance of the user and providing this information to the user. It will be appreciated, however, that the device could be arranged to be carried by a user or connected or “docked” in a known manner to a vehicle such as a bicycle, kayak, or the like.

In general, GPS is a satellite-radio based navigation system capable of determining continuous position, velocity, time, and in some instances direction information for an unlimited number of users. Formerly known as NAVSTAR, the GPS incorporates a plurality of satellites which orbit the earth in extremely precise orbits. Based on these precise orbits, GPS satellites can relay their location to any number of receiving units.

The GPS system is implemented when a device, specially equipped to receive GPS data, begins scanning radio frequencies for GPS satellite signals. Upon receiving a radio signal from a GPS satellite, the device determines the precise location of that satellite via one of a plurality of different conventional methods. The device will continue scanning, in most instances, for signals until it has acquired at least three different satellite signals (noting that position is not normally, but can be determined, with only two signals using other triangulation techniques). Implementing geometric triangulation, the receiver utilizes the three known positions to determine its own two-dimensional position relative to the satellites. This can be done in a known manner. Additionally, acquiring a fourth satellite signal will allow the receiving device to calculate its three dimensional position by the same geometrical calculation in a known manner. The position and velocity data can be updated in real time on a continuous basis by an unlimited number of users.

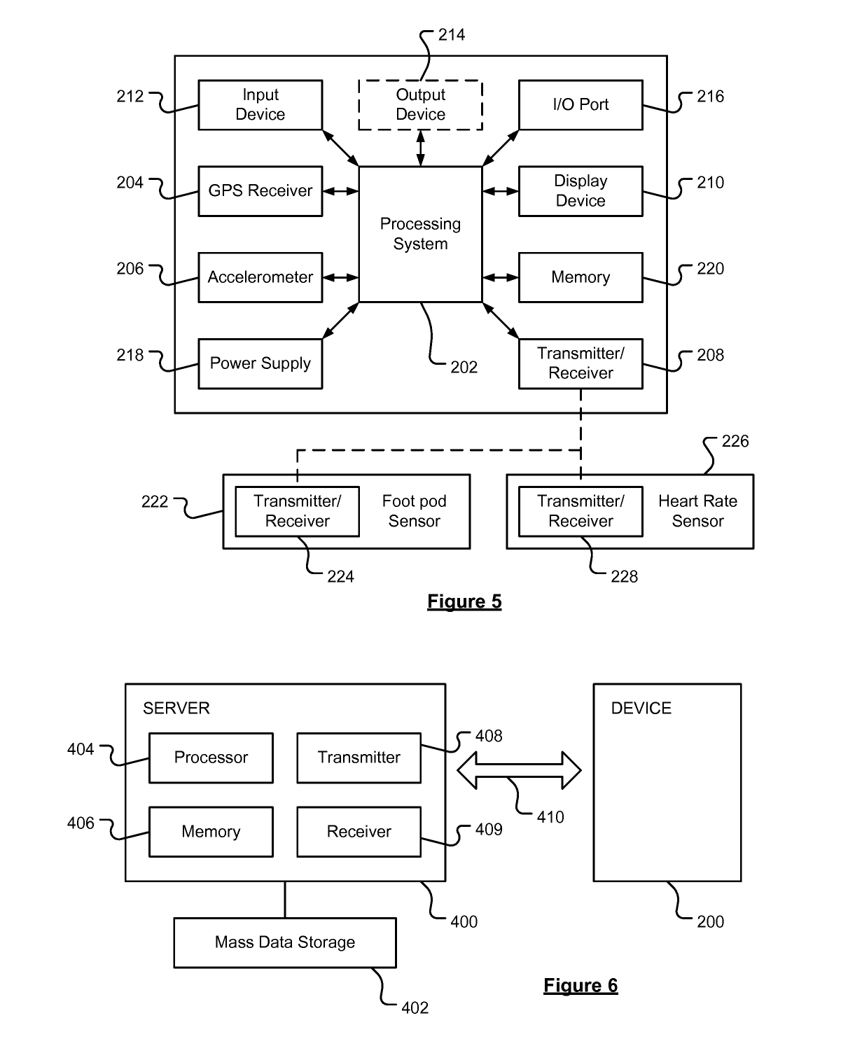

FIG. 5 is an illustrative representation of electronic components of a sports watch 200 according to a preferred embodiment of the present invention, in block component format. It should be noted that the block diagram of the device 200 is not inclusive of all components of the device, but is only representative of many example components.

The device 200 includes a processor 202 connected to an input device 212, such as a depressible touchpad (or trackpad), and a display screen 210, such as an LCD display. The device 200 can further include an output device arranged to provide audible information to a user, such as alerts that a certain speed has been reached or a certain distance has been travelled.

FIG. 5 further illustrates an operative connection between the processor 202 and a GPS antenna/receiver 204. Although the antenna and receiver are combined schematically for illustration, the antenna and receiver may be separately located components. The antenna may be of any suitable form, but in preferred embodiments is a GPS patch antenna.

The device 200 further includes an accelerometer 206, which can be a 3-axis accelerometer arranged to detect accelerations of the user in x, y and z directions. The accelerometer may act as a pedometer for use when/if there is a loss of GPS reception, and/or may act to detect stroke rate when the fitness watch is being used during swimming. Although the accelerometer is shown to be located within the device, the accelerometer may also be an external sensor worn or carried by the user, and which transmits data to the device 200 via the transmitter/receiver 208.

The device may also receive data from other sensors, such as a foot pod sensor 222 or a heart rate sensor 226. The foot pod sensor may, for example, be a piezoelectric or micro-electro-mechanical systems (MEMS) accelerometer that is located in or on the sole of the user's shoe. Each external sensor is provided with a transmitter and receiver, 224 and 228 respectively, which can be used to send or receive data to the device 200 via the transmitter/receiver 208.

The processor 202 is operatively coupled to a memory 220. The memory resource 220 may comprise, for example, a volatile memory, such as a Random Access Memory (RAM), and/or a non-volatile memory, for example a digital memory, such as a flash memory. The memory resource 220 may be removable. As discussed in more detail below, the memory resource 220 is also operatively coupled to the GPS receiver 204, the accelerometer 206 and the transmitter/receiver 208 for storing data obtained from these sensors and devices.

Further, it will be understood by one of ordinary skill in the art that the electronic components shown in FIG. 5 are powered by a power source 218 in a conventional manner. The power source 218 may be a rechargeable battery.

The device 200 further includes an input/output (I/O) device 216, such as a plurality of electrical contacts or a USB connector. The I/O device 216 is operatively coupled to the processor, and also at least to the memory 220 and power supply 218. The I/O device 216 is used, for example, to: update firmware of processor 220, sensors, etc; transfer data stored on the memory 220 to an external computing resource, such as a personal computer or a remote server; and recharge the power supply 218 of the device 200. Data could, in other embodiments, also be sent or received by the device 200 over the air using any suitable mobile telecommunication means.

As will be understood by one of ordinary skill in the art, different configurations of the components shown in FIG. 5 are considered to be within the scope of the present application. For example, the components shown in FIG. 5 may be in communication with one another via wired and/or wireless connections and the like.

In FIG. 6 the watch 200 is depicted as being in communication with a server 400 via a generic communications channel 410 that can be implemented by any number of different arrangements. The server 400 and device 200 can communicate when a connection is established between the server 400 and the watch 200 (noting that such a connection can be a data connection via mobile device, a direct connection via personal computer via the internet, etc.).

The server 400 includes, in addition to other components which may not be illustrated, a processor 404 operatively connected to a memory 406 and further operatively connected, via a wired or wireless connection, to a mass data storage device 402. The processor 404 is further operatively connected to transmitter 408 and receiver 409, to transmit and send information to and from device 200 via communications channel 410. The signals sent and received may include data, communication, and/or other propagated signals. The functions of transmitter 408 and receiver 409 may be combined into a signal transceiver.

The communication channel 410 is not limited to a particular communication technology. Additionally, the communication channel 410 is not limited to a single communication technology; that is, the channel 410 may include several communication links that use a variety of technology. For example, the communication channel 410 can be adapted to provide a path for electrical, optical, and/or electromagnetic communications, etc. As such, the communication channel 410 includes, but is not limited to, one or a combination of the following: electric circuits, electrical conductors such as wires and coaxial cables, fibre optic cables, converters, radio-frequency (RF) waves, the atmosphere, empty space, etc. Furthermore, the communication channel 410 can include intermediate devices such as routers, repeaters, buffers, transmitters, and receivers, for example.

In one illustrative arrangement, the communication channel 410 includes telephone and computer networks. Furthermore, the communication channel 410 may be capable of accommodating wireless communication such as radio frequency, microwave frequency, infrared communication, etc. Additionally, the communication channel 410 can accommodate satellite communication.

The server 400 may be a remote server accessible by the watch 200 via a wireless channel. The server 400 may include a network server located on a local area network (LAN), wide area network (WAN), virtual private network (VPN), etc.

The server 400 may include a personal computer such as a desktop or laptop computer, and the communication channel 410 may be a cable connected between the personal computer and the watch 200. Alternatively, a personal computer may be connected between the watch 200 and the server 400 to establish an internet connection between the server 400 and the watch 200. Alternatively, a mobile telephone or other handheld device may establish a wireless connection to the internet, for connecting the watch 200 to the server 400 via the internet.

The server 400 is further connected to (or includes) a mass storage device 402. The mass storage device 402 contains a store of at least digital map information. This digital map information can be used, together with data from the device, such as time-stamped location data obtained from the GPS receiver 204 and data indicative of motion of the wearer obtained from the accelerometer 206, footpad sensor 222, etc, to determine a route travelled by the wearer of the device 200, which can then be viewed by the wearer.

As will be appreciated, the watch 200 is designed to be worn by a runner or other athlete as they undertake a run or other similar type of workout. The various sensors within the watch 200, such as the GPS receiver 204 and the accelerometer 206, collect data associated with this run, such as the distance travelled, current speed, etc, and display this data to the wearer using the display screen 210.

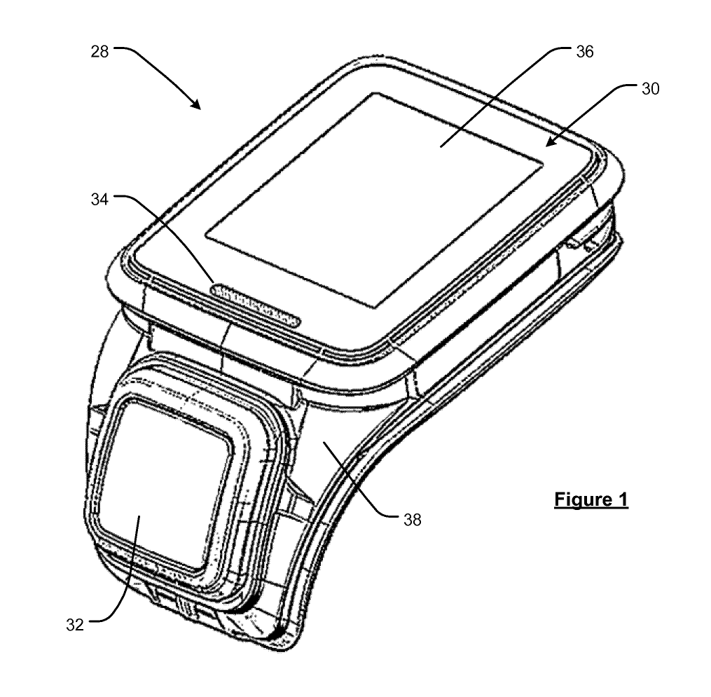

FIGS. 1 to 4 provide an example of a fitness monitoring module that can be removably connected to a wrist strap (not shown), such as one as discussed above. In some embodiments the module 28 could take the form of a fitness watch module, in particular a GNSS, e.g. GPS, watch module. In other embodiments the module 28 takes the form of a heart rate monitor module, which may or may not include GNSS watch capabilities.

FIG. 1 shows a perspective view of a heart rate monitor module 28 comprising a housing 30 and a display 36 exposed at an upper surface of the housing 30. An input device 32 is spaced apart from the display 36. The substantially planar display 36 is controlled by the input device 32 to display heart rate information to a user. The display 36 may comprise a liquid crystal display (LCD). In addition to the LCD, the display includes an illumination 34, for example an LED that shines through the otherwise opaque frame of the LCD.

The illumination 34 is controlled by a processor in the watch module 28 to convey information relating to a user's heart rate. In one example, the illumination 34 blinks at the approximate frequency of the heart rate, thereby providing for visualisation of the heart rate at a glance. In addition, or alternatively, in another example the colour of the illumination 34 represents a particular heart rate zone. The illumination 34 may blink according to the following colour scheme:

| HEART RATE ZONE | COLOUR |
|---|---|
| 1. Recover | Turquoise |
| 2. Fat Burn | Blue |
| 3. Endure | Green |
| 4. Speed | Purple |
| 5. Sprint | Red |

The input device 32 is connected to the main housing 30 by a curved flange 38 that extends away from the housing 30. The curved flange 38 extends away from the housing 30 such that it curves around a user's wrist when the module is mounted to a wrist strap (not shown). The input device 32 is located so as to be arranged on the side of the user's wrist in use. The input device 32 has a substantially planar pressing surface for the user to interact with the module 28. The user can thereby press the pressing surface in a direction perpendicular to the pressing surface so as to control the module 28, e.g. to select desired functions within the menu system of the heart rate monitor. In this example the input device 32 takes the form of a four-way button.

The location of the input device 32 being arranged on the curved flange 38 such that it sits against the side of the user's wrist in use has a number of important advantages. For example, this enables the user to interact with the module 28 using only a single finger. More specifically, the user is able to push the pressing surface of the input device 32 with one finger because the user pushes the surface into the user's wrist around which the watch 28 is strapped. This is in contrast to conventional heart rate monitors or watches wherein buttons are arranged around the peripheral edges and the user must press the button with on finger and use a thumb on the other edge of the watch to counter-balance the pressing force. As seen in FIG. 1, for example, the plane defined by the substantially planar display 36 is arranged at an angle to the plane defined by the input device 32, the dihedral angle between the two planes being less than 90 degrees, and typically between 20 and 70 degrees.

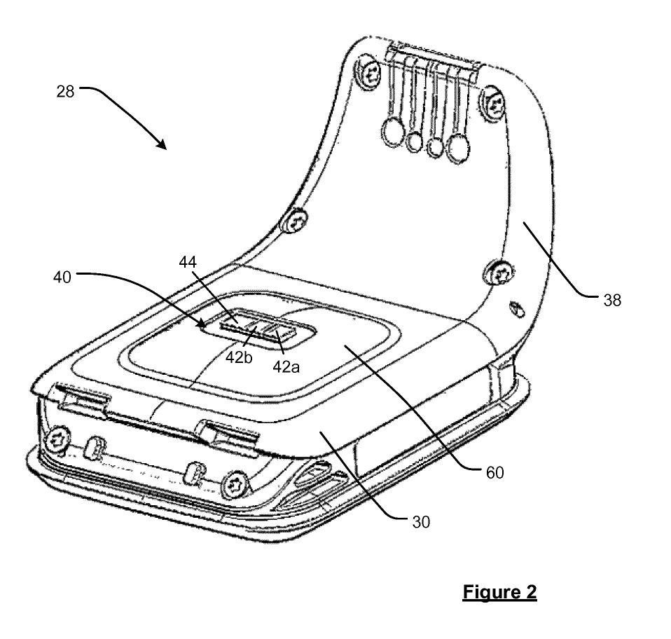

FIG. 2 shows a perspective view of the heart rate module 28 from the underside. The curved flange 38 that extends from the main housing 30 may have electrical connectors (not seen) arranged at a distal end thereof. These electrical connectors may be used in order to electrically connect the module 28 to a dock in order to recharge a battery within the module 28 and/or to extract data from or input data to the module 28. The lower surface of the housing 30 comprises a domed portion 60 that extends over an optical heart rate (OHR) sensor. A sensing unit 40 of the OHR sensor protrudes through an aperture in the domed portion 60. In this example the sensing unit 40 comprises a pair of light emitting diodes 42a, 42b (e.g. two green LEDs, or a green LED and an infrared LED) and a photodetector 44. When the module 28 is mounted to a wearer's wrist, the sensing unit 40 sits on the front or back of the wrist in contact with the skin. The domed portion 60 applies pressure so that the module 28 is less likely to move when it is worn, while the protrusion of the sensing unit 40 presses into the skin so as to prevent or reduce the ingress of ambient light to the photodetector 44.

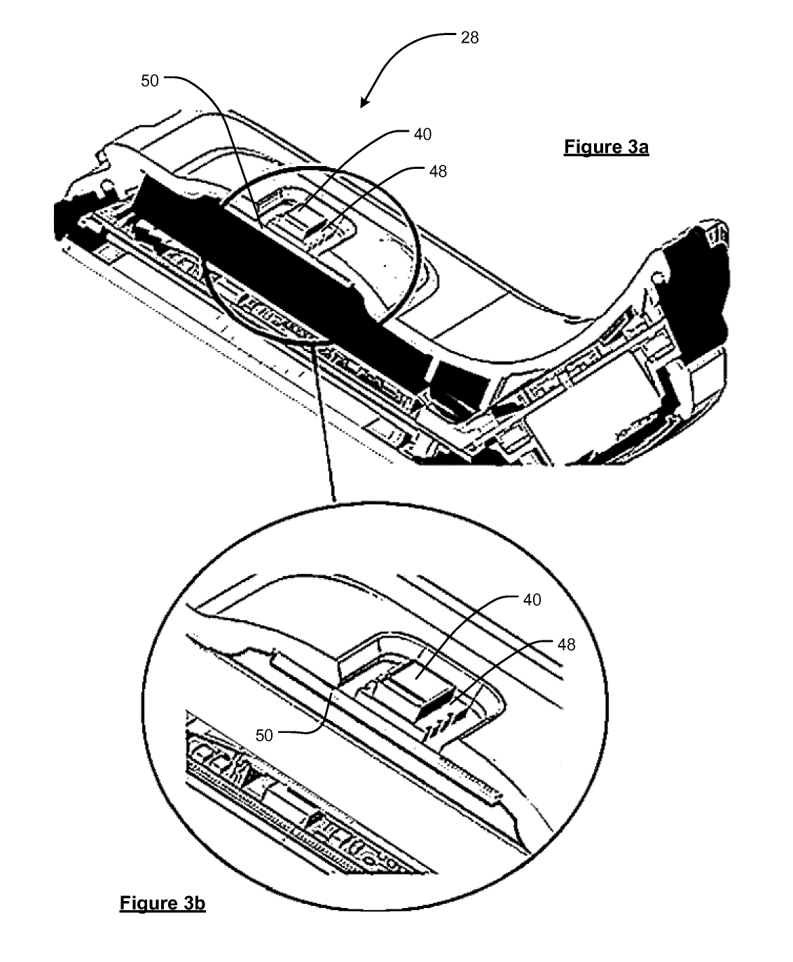

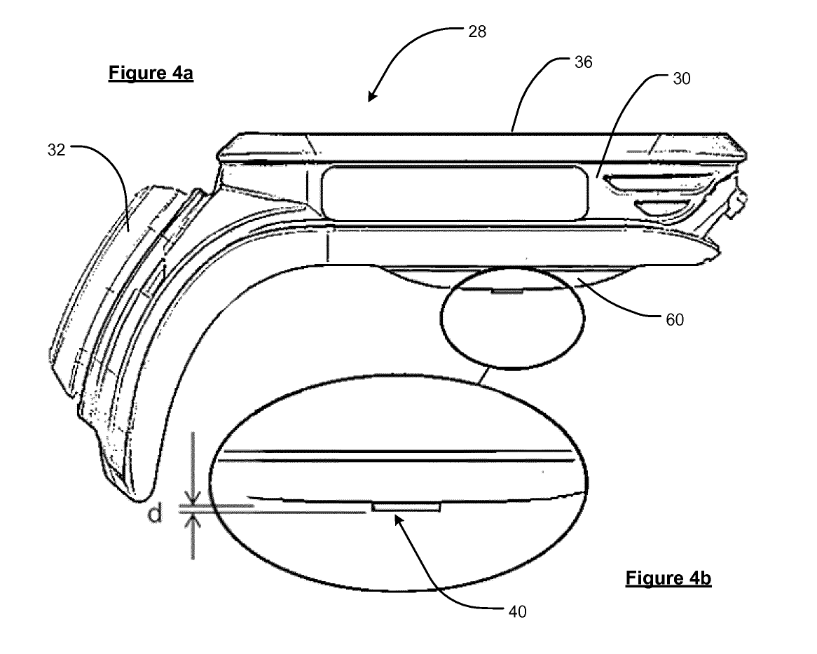

FIGS. 3a and 3b show in detail how the sensing unit 40 is mounted on a riser 48 on top of a printed circuit board 50. The depth of the riser 48 determines how far the sensing unit 40 protrudes beyond the surface of the domed portion 60. The side view of FIGS. 4a and 4b shows how the sensing unit 40 protrudes from the surface of the domed portion 60 by a distance d. The distance d is at least 0.1 mm.

The OHR sensing unit 40 may be operatively connected to a processor in the module 28 that can process data signals relating to pulse and/or heart rate. The processor is typically connected to a memory and a power supply, e.g. a battery. The battery may be recharged when the module 28 is docked using its I/O port, for example in the form of the electrical connectors mentioned above. The same electrical connectors may also be used to transfer data to/from the processor. In addition to the I/O port, the module 28 may include a wireless communications interface, such as a Bluetooth transceiver, that enables the module 28 to wirelessly communicate with one or more other devices to receive additional data. For example, other devices that are body-worn (e.g. an external heart rate monitor) or mounted nearby during exercise (e.g. mounted to a bike during cycling activities) may pair with the module 28 to transmit additional data. The module's user interface may allow a user to view such additional data on the display. In some embodiments the module 28 could take the form of a fitness watch module, in particular a GNSS, e.g. GPS, watch module.

The module's user interface includes the display 36 and the input device 32 already described above. Of course other user interface components may be provided instead, or as well as, those seen in the figures. Further features of a module 28 as seen in FIGS. 1 to 4 are described in WO 2014/135709; the contents of which are hereby incorporated by reference. In particular, it is described therein how such a module may be removably mounted to a wrist strap.

It will be appreciated that whilst various aspects and embodiments of the present invention have heretofore been described, the scope of the present invention is not limited to the particular arrangements set out herein and instead extends to encompass all arrangements, and modifications and alterations thereto, which fall within the scope of the appended claims.

For example, whilst a preferred embodiment described in the foregoing detailed description relates to a heart rate monitor module without reference to a strap, it will be understood that the module could be mounted to a wrist strap, either permanently or removably. Furthermore, although the module has been described as having a display and/or input device, these are optional components. A suitable module may include a battery and a processor connected to one or more of: an optional display, an optional input device, a memory, a wireless transceiver, and an input/output device such as electrical contacts.

Lastly, it should be noted that whilst the accompanying claims set out particular combinations of features described herein, the scope of the present invention is not limited to the particular combinations hereafter claimed, but instead extends to encompass any combination of features or embodiments herein disclosed irrespective of whether or not that particular combination has been specially enumerated in the accompanying claims at this time.

1. A heart rate monitor comprising a housing and an optical heart rate (OHR) sensor within the housing, the OHR sensor comprising a sensing unit including at least one light emitter arranged to emit light into the skin of a user and a photodetector arranged to sense light reflected through the skin of the user, wherein the housing comprises a domed portion, the sensing unit being exposed through a surface of the domed portion, and wherein the sensing unit protrudes above the surface of the domed portion.

2. The heart rate monitor of claim 1, wherein the sensing unit protrudes above the surface of the domed portion by at least 0.1 mm.

3. The heart rate monitor of claim 2, wherein the sensing unit protrudes above the surface of the domed portion by up to 0.8 mm and preferably up to 0.5 mm.

4. The heart rate monitor of claim 1, wherein the sensing unit is mounted on a circuit board by a riser.

5. The heart rate monitor of claim 1, further comprising a sealant around the sensing unit.

6. The heart rate monitor of claim 5, wherein the sensing unit is exposed through an aperture in the surface of the domed portion and the sealant fills the aperture.

7. The heart rate monitor of claim 5, wherein the sealant comprises epoxy resin.

8. The heart rate monitor of claim 1, wherein the sensing unit comprises three light emitting diodes of different wavelengths.

9. The heart rate monitor of claim 1, wherein the sensing unit is fully integrated as a package having a depth of less than 1 mm.

10. The heart rate monitor of claim 1, further comprising a display for displaying heart rate information to a user.

11. The heart rate monitor of claim 1, further comprising one or more illumination devices in addition to the display, and wherein the one or more illumination devices are controlled: (i) so as to flash at a frequency that is dependent on heart rate information; and/or (ii) so that the colour of emitted light corresponds to a predetermined heart rate zone.

12. A watch, comprising the heart rate monitor comprising a housing and an optical heart rate (OHR) sensor within the housing, the OHR sensor comprising a sensing unit including at least one light emitter arranged to emit light into the skin of a user and a photodetector arranged to sense light reflected through the skin of the user, wherein the housing comprises a domed portion, the sensing unit being exposed through a surface of the domed portion, and wherein the sensing unit protrudes above the surface of the domed portion and a strap for securing the watch to the arm or wrist of a user.

13. The watch of claim 12, wherein the watch is a fitness watch.
